(19) United States
(12) Patent Application Publication
(10) Pub. No.: US 2022/0079883 A1
(43) Pub. Date: Mar. 17, 2022

(54) ELTROMBOPAG CHOLINE DOSAGE FORMS

(71) Applicant: ACTAVIS LABORATORIES FL, INC., Davie, FL (US)

(72) Inventors: Alap CHOUDHARI, Davie, FL (US); Ashok Kumar THALLUR GOPI, Davie, FL (US); Soumen PATTANAYEK, Davie, FL (US); Parag SHAH, Davie, FL (US); Mayank JOSHI, Davie, FL (US)

(21) Appl. No.: 17/472,783

(22) Filed: Sep. 13, 2021

Related U.S. Application Data
(60) Provisional application No. 63/078,115, filed on Sep. 14, 2020.

(51) Int. Cl. A61K 9/20; A61K 9/00; A61K 31/4152

(52) U.S. Cl. CPC A61K 9/2095; A61K 9/0053; A61K 31/4152; A61K 9/2054; A61K 9/2027; A61K 9/2018; A61K 9/2031

(57) ABSTRACT

Oral dosage forms comprising eltrombopag choline, processes for preparation thereof and methods of use thereof are disclosed. The compositions disclosed exhibit enhanced bioavailability and reduced food effect compared to commercially available formulations of eltrombopag.

### CROSS REFERENCE TO RELATED APPLICATION

This application claims the benefit of U.S. Provisional Patent Application No. 63/078,115 filed Sep. 14, 2020, the entirety of which is incorporated by reference herein.

### FIELD OF THE INVENTION

The present disclosure relates to eltrombopag choline oral dosage forms, processes for preparation thereof and uses thereof.

### BACKGROUND OF THE INVENTION

Eltrombopag choline, chemical name 3′-(2-(1-(3,4-dimethylphenyl)-3-methyl-5-oxo-1,5-dihydro-4H-pyrazol-4-ylidene)hydrazineyl)-2′-hydroxy-[1,1′-biphenyl]-3-carboxylic acid 2-hydroxy-N,N,N-trimethylethan-1-aminium (1:1), has the following chemical structure:

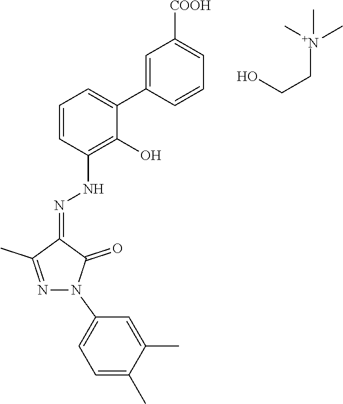

Eltrombopag is a small-molecule, non-peptide thrombopoietin (TPO) receptor agonist that has been shown to stimulate the proliferation and differentiation of megakaryocytes, the bone marrow cells that give rise to blood platelets in pre-clinical research and clinical trials.

Eltrombopag bisethanolamine is the active ingredient of the marketed drug Promacta® (US) or Revolade® (EU) indicated in the treatment of chronic immune thrombocytopenia (CIT) in patients who have had an insufficient response to corticosteroids, immunoglobulins, or splenectomy. It is also indicated for thrombocytopenia in patients with chronic hepatitis C and in severe aplastic anemia.

Eltrombopag is disclosed in U.S. Pat. No. 7,160,870. Eltrombopag bisethanolamine salt is disclosed in U.S. Pat. No. 7,547,719. Eltrombopag tromethamine salt is disclosed in International Patent Publication No. WO20170042839. Crystalline forms of eltrombopag choline are disclosed in International Patent Publication No. WO20190071111 and U.S. Pat. No. 11,072,586.

There is an unmet need for an eltrombopag drug product that exhibits beneficial features for patients, including maintaining a therapeutic effect at lower doses and having a reduced food effect than the commercially available product.

### SUMMARY OF THE INVENTION

The present disclosure relates to oral dosage forms of eltrombopag choline, processes for preparation thereof, and methods of treatment using these dosage forms.

Provided herein is a solid dispersion (e.g. granules) of eltrombopag choline and at least one hydrophilic polymer. Further provided is an oral dosage form comprising the solid dispersion comprising eltrombopag choline and at least one hydrophilic polymer. In some embodiments, the at least one hydrophilic polymer is selected from a poloxamer, polyvinylpyrrolidone, a polyethylene glycol (PEG) and any combination thereof. The preferred at least one hydrophilic polymer has a molecular weight (MW) in the range of about 2,000 to about 80,000 daltons, or about 3,000 to about 75,000 daltons. In some embodiments, the dosage form may comprise a combination of hydrophilic polymers, for example any mixture of a poloxamer, a polyvinylpyrrolidone and a polyethylene glycol. In some embodiments, the dosage form comprises a mixture of a low MW hydrophilic polymer and a mid-weight MW hydrophilic polymer. For example, at least one hydrophilic polymer has a molecular weight in the range of about 2,000 to about 10,000 daltons (low MW) and at least one second hydrophilic polymer has a molecular weight in the range of about 30,000 to about 80,000 daltons (mid-weight MW). In some embodiments, the low MW hydrophilic polymer is a poloxamer, PEG, povidone or a mixture thereof. In some embodiments, the mid-weight MW hydrophilic polymer is copovidone. The total amount of the at least one hydrophilic polymer, or combination of polymers, is present in the dosage form at a concentration of about 15 wt % to about 60 wt % of the total weight of the dosage form. In some embodiments, the eltrombopag choline is present at a concentration of about 5 wt % to about 30 wt % of the total weight of the dosage form. The ratio of eltrombopag choline to total hydrophilic polymer in the dosage form is about 1:1 to about 1:5, or about 1:1 to about 1:4, or about 1:2 to about 1:3, or about 1:2. In some embodiments, the eltrombopag choline solid dispersion is prepared by hot-melt extrusion.

In addition to the eltrombopag choline and the at least one hydrophilic polymer, the oral dosage form further comprises at least one excipient. In some embodiments, the at least one excipient may be selected from a disintegrant, a lubricant, a filler, a chelating agent and any combination thereof. In some embodiments, the oral dosage form includes a disintegrant, the disintegrant selected from hydroxypropyl cellulose (HPC), carboxymethyl cellulose and salts thereof, croscarmellose sodium, magnesium aluminum silicate, sodium starch glycolate, a starch or any combination thereof. The starch includes a food starch which may be, for example, corn starch, potato starch, tapioca starch and the like. The disintegrant may be present in the dosage form at a concentration of about 3 wt % to about 25 wt %, of the total weight of the dosage form.

In some embodiments, the oral dosage form includes a lubricant, the lubricant selected from magnesium stearate, stearic acid, sodium stearyl fumarate, calcium stearate, hydrogenated vegetable oil, mineral oil, talc, and glyceryl behenate, or any combination thereof. The lubricant may be present in the dosage form at a concentration of about 0.05 wt % to about 2.5 wt %, of the total weight of the dosage form.

In some embodiments, the oral dosage form includes a filler, the filler selected from one or more of mannitol, microcrystalline cellulose, silicified microcrystalline cellulose, anhydrous lactose, dicalcium phosphate, tricalcium phosphate, starch, pregelatinized starch, compressible sugars, calcium carbonate or any combination thereof. The filler may be present in the dosage form at a concentration of about 30 wt % to about 70 wt %, of the total weight of the dosage form.

The oral dosage form optionally includes a chelator, the chelator selected from one or more of ethylenediaminetetraacetate (EDTA), ammonium citrate dibasic, calcium citrate, calcium disodium ethylenediaminetetraacetate (calcium disodium EDTA), disodium ethylenediaminetetraacetate (disodium EDTA), glycine, sodium citrate, sodium hexametaphosphate, stearyl citrate, trisodium phosphate or any combination thereof. The chelator may be present in the dosage form at a concentration of about 0.05 wt % to about 10 wt % of the total weight of the dosage form. In certain embodiments, the oral dosage form comprises a disintegrant, a lubricant, a chelating agent and a filler.

In some embodiments the oral dosage form comprises (wt % of total weight)
from about 5 wt % to about 30 wt % of eltrombopag choline;
from about 15 wt % to about 60 wt % of one or more hydrophilic polymers; and one or more excipient.

In some embodiments, the oral dosage form comprises an excipient which is a disintegrant, in an amount of from about 3 wt % to about 25 wt %;

In some embodiments, the oral dosage form comprises an excipient which is a filler, in an amount of from about 30 wt % to about 70 wt %;

In some embodiments, the oral dosage form comprises an excipient which is a lubricant, in an amount of from about 0.05 wt % to about 2.5 wt %;

In some embodiments the oral dosage form comprises an excipient which is a chelator, in an amount of from about 0.05 wt % to about 10 wt %.

In some embodiments, the oral dosage form comprises
from about 10 wt % to about 15 wt % of eltrombopag choline;
from about 15 wt % to about 30 wt % of one or more hydrophilic polymers;
from about 40 wt % to about 50 wt % of one or more fillers;
from about 10 wt % to about 15 wt % of one or more disintegrants;
from about 0.1 wt % to about 1 wt % of one or more lubricants; and
from about 0.5 wt % to about 10 wt % of at least one chelating agent.

The oral dosage forms may be solid dosage units, including pills and tablets, or may be processed into capsules. By means of pharmaceutically suitable liquids, the oral dosage forms may be administered in the form of a solution, suspension or emulsion. In some embodiments, the oral dosage forms disclosed herein are presented in the form of a tablet, or granules, for example in a sachet package or as a liquid suspension. In some embodiments, the oral dosage form disclosed herein is presented as a tablet. In some embodiments, the oral dosage form disclosed herein is presented as granules. In some embodiments, the oral dosage form disclosed herein is presented as an oral suspension. In some embodiments, the oral dosage form comprises 9 mg eltrombopag choline, 18 mg eltrombopag choline, 25 mg eltrombopag choline, 36 mg eltrombopag choline, 50 mg eltrombopag choline, 54 mg eltrombopag choline or 72 mg eltrombopag choline. In some preferred embodiments, the oral dosage form comprises 9 mg eltrombopag choline, 18 mg eltrombopag choline, 36 mg eltrombopag choline, 54 mg eltrombopag choline or 72 mg eltrombopag choline.

Further provided are uses for the oral dosage forms and methods of treating using the oral dosage forms. In some embodiments, the oral dosage forms are useful in treating a disorder in which stimulating the proliferation and differentiation of megakaryocytes provides a beneficial effect on a subject suffering from such a disorder. In some embodiments, provided is a method of treating a subject having a disorder in which stimulating the proliferation and differentiation of megakaryocytes provides a beneficial effect on the subject, the method comprising administering an oral dosage form disclosed herein to a subject suffering from the disorder. In some embodiments of the use and method, the oral dosage form may be administered to a subject without regard to food. In some embodiments, the disorder is idiopathic thrombocytopenic purpura. In some embodiments, the disorder is thrombocytopenia. In some embodiments, the disorder is aplastic anemia.

In some embodiments, the oral dosage forms exhibit at least one of an increase in Cmax, AUC0-72 or AUC0-inf by at least 25%, or by at least 30% when orally administered to a subject under fasting conditions compared to an equivalent dose of the marketed eltrombopag olamine product (Promacta®).

Further provided is a process for preparing eltrombopag choline granules, comprising:
a. providing eltrombopag choline and at least one hydrophilic polymer
b. mixing the eltrombopag choline and the at least one hydrophilic polymer to form a mixture;
c. heating the mixture of step (b) to a temperature sufficient to form a molten dispersion of the eltrombopag choline and the at least one hydrophilic polymer;
d. milling the dispersion from step (c) thereby providing the eltrombopag choline granules.

The eltrombopag choline granules may be used to prepare the eltrombopag choline dosage forms, for example by admixing with one or more excipient.

In some embodiments, the method further comprises the following step
e. mixing the granules from step (d) with a common blend to form an eltrombopag choline blend.

In various embodiments, the method further comprises the steps of
f. compressing the eltrombopag choline blend of step (e) into tablets, or filling a capsule shell or filling a sachet package with the eltrombopag choline blend of step (e); and
g. optionally, coating the tablets or capsule shell;
thereby providing an eltrombopag oral dosage form.

In various embodiments the at least one hydrophilic polymer comprises Poloxamer 188, Copovidone, Povidone, PEG 4000 or any combination thereof. The blend may be compressed into solid dosage units, such as pills, tablets, including mini tablets or be processed into capsules. By means of pharmaceutically suitable liquids the blends may also be provided in the form of a solution, suspension, or emulsion. In some embodiments, the composition of the common blend is appropriate for manufacture of an eltrombopag choline tablet. In some embodiments, the composition of the common blend is appropriate for manufacture of an eltrombopag choline suspension. The composition of such blends are well known among those of ordinary skill in the art. In some embodiments, the common blend includes at least one filler, at least one disintegrant, at least one lubricant, at least one chelating agent or any combination thereof. In some embodiments, the common blend includes a mixture of at least one filler, at least one disintegrant, at least one lubricant and at least one chelating agent.

Further provided are eltrombopag choline granules comprising a mixture of eltrombopag and at least one hydrophilic polymer. The hydrophilic polymer may be selected from a poloxamer, polyvinylpyrrolidone, a polyethylene glycol and any combination thereof, or a mixture of a poloxamer, polyvinylpyrrolidone, and a polyethylene glycol.

### BRIEF DESCRIPTION OF THE FIGURES

FIG. 1 provides a flow chart for manufacture of an exemplary eltrombopag choline composition.

FIG. 2 shows a release profile of eltrombopag choline oral dosage forms disclosed herein.

FIG. 3 shows a semi-log plot of mean plasma eltrombopag concentrations vs. time.

### DETAILED DESCRIPTION OF THE INVENTION

The present disclosure relates to oral dosage forms of eltrombopag choline, processes for preparation thereof and uses thereof.

The compositions according to the present disclosure may have advantageous properties including increased bioavailability and/or reduced food effect, when compared to the commercially available formulations.

The term “therapeutically effective amount” refers to the amount of a compound, e.g. eltrombopag choline, that, when administered in the oral dosage form disclosed herein, is sufficient to elicit the biological or medical response of a cell, tissue, system, animal, or human that is being sought by a clinician.

The terms “subject” and “patient” are used interchangeably herein, for example, to a mammalian subject, preferably a human or human patient.

The singular forms “a,” “an,” and “the” may refer to plural articles unless specifically stated otherwise.

The term “about,” as used herein, is intended to qualify the numerical values which it modifies, denoting such a value as variable within a margin of ±10%.

The term “combination therapy” refers to the administration of two or more therapeutic agents to treat a therapeutic disorder described herein. Such administration encompasses co-administration of these therapeutic agents in a substantially simultaneous manner, such as in a single dosage form having a fixed ratio of active ingredients or in multiple, separate dosage forms for each active ingredient. In addition, such administration also encompasses use of each type of therapeutic agent in a sequential manner. In either case, the treatment regimen will provide beneficial effects of the drug combination in treating the disorders described herein. An example is in the treatment of severe aplastic anemia, wherein eltrombopag in combination with standard immunosuppressive therapy is useful for the first-line treatment of adult and pediatric patients 2 years and older (Townsley et al, 2017. NEJM, 376:1540-50). Standard immunosuppressive therapy includes, inter alia, antithymocyte globulin and cyclosporine. The uses and methods disclosed herein encompass combination therapy.

As used herein “eltrombopag choline” refers to the choline salt of eltrombopag. In principle, any crystalline form or amorphous form of eltrombopag choline may be used for the preparation of the oral dosage forms disclosed herein. In some embodiments, the oral dosage forms are manufactured by hot melt extrusion. In some embodiments, the oral dosage forms are manufactured by hot melt extrusion and may comprise eltrombopag choline amorphous form.

According to the Food and Nutrition Board of the National Institute of Medicine, the adequate intake for choline is 200-600 mg daily for adults and for children between 125-375 mg/day depending on age. For eltrombopag choline, equivalent intake of the 588.3 mg/day of the counterion is well tolerable.

The eltrombopag choline oral dosage forms disclosed herein exhibit improved absorption of eltrombopag and increased bioavailability of the drug and reduced food effect in comparison to the commercially available eltrombopag olamine oral tablets (Promacta®). Furthermore, the oral dosage forms according to the invention are prepared by a hot melt extrusion process, which is easy to control and has good yield and productivity.

An “oral dosage form” and an “oral pharmaceutical composition” are used interchangeably herein. The dosage forms disclosed herein comprise eltrombopag choline and one or more hydrophilic carrier, which may be hydrophilic polymers. In some embodiments, the dosage form comprises eltrombopag choline dispersed in the one or more hydrophilic polymers. In some embodiments, the eltrombopag choline-polymer dispersion is a molten dispersion comprising eltrombopag choline and at least one hydrophilic polymer. The molten dispersion may result from a hot melt extrusion process.

The term “hot melt extrusion” refers to the process of forming an extrudate which includes one or more polymer and one or more active pharmaceutical ingredient (API) under heat and compression. Without wishing to be bound to theory, the process results in the dispersion of the API, for example, the eltrombopag choline, into a polymer matrix to form an extrudate. An “extrudate” refers to a hot melt extruded composition. In some embodiments, for example in cases where the eltrombopag choline-polymer molten dispersion results from a hot melt extrusion process, the molten dispersion is also referred to as an extrudate. A suitable extruder will be selected by the person with skill in the art. Typical extrudes include single-screw extruders (SSEs) with a smooth or grooved barrel; twin-screw extruders (TSEs) which are co-rotating or counter-rotating with or without intermeshing screws; and multi-screw extruders (MSEs) with a static or rotating central shaft. The extrudates can be in any form or shape including beads, granules, tubes or cylinders and may be further processed into any desired shape. In some embodiments, the extrudate is further processed, for example by milling, to provide a particulate or granulate form of the eltrombopag choline and the at least one hydrophilic polymer. Such form may also be referred to as a “solid dispersion” of eltrombopag choline.

The pharmacokinetic terms “Cmax”, “AUC₀₋₇₂” or “AUC_{0-inf}” are terms known in the art and refer to maximum observed concentration, the area under the plasma concentration versus time curve from time zero to 72 hours, and area under the concentration-time curve from time zero to infinity, respectively.

The term “bioavailability” or abbreviated as “BA”, refers to the amount of a drug, i.e. eltrombopag or salt thereof which is absorbed in the body and is able to have an effect. For example, bioavailability may refer to the fraction of drug in systemic circulation following administration to a subject or patient under fed or fasted state.

It is well understood in the art of pharmaceutical formulation that the pharmacokinetic (PK) performance of some compositions is affected by the presence or absence of food in the gastrointestinal (GI) system. A “food effect study” is typically undertaken to observe the effect of food on drug bioavailability (BA) between fed and fasted treatments. Accordingly, administration of an oral dosage form exhibiting a food effect may preferably be made under “fasted” conditions, for example 1 hour before or 2 hours after a meal. As used herein, the term “without regard to food” or “without regard to meals” means that the human exposure to the drug is not affected by food and that the drug product, i.e. eltrombopag choline oral dosage form, may be taken either on an empty stomach or irrespective of the human subject's fed state.

The US FDA requirements for a food effect study, and definition of a fasted state and “fed state” may be found at https://www.regulations.gov/document?D=FDA-2001-D-0040-0003 (Jul. 22, 2020), the entirety of which is incorporated herein by reference.

The at least one “hydrophilic polymer” as used herein is a polymer having hydrophilic properties, i.e. soluble in water and/or able to absorb water, often due to polar or charged functional groups. Without wishing to be bound to theory, the hydrophilic polymer is able to modify the release profile of an API from an oral dosage form, compared to, for example an immediate release oral dosage form. In preferred embodiments, “modified release” refers to a delayed release or extended release compared to an immediate release. In some embodiments, a single type of hydrophilic polymer is included in the dosage form. In other embodiments a plurality of hydrophilic polymers are included in the dosage form. The preferred at least one hydrophilic polymer has a molecular weight (MW) in the range of about 2,000 to about 80,000 daltons, or about 3,000 to about 75,000 daltons. In some embodiments, the dosage form comprises at least two different hydrophilic polymers. In some embodiments, the at least two different hydrophilic polymers comprise hydrophilic polymers of different MW. For example, one of the polymers may be a low MW hydrophilic polymer, which may be a poloxamer, a povidone or a PEG preferably having a MW of about 2,000 to about 10,000 dalton. A second polymer may be a mid-weight hydrophilic polymer having a MW in the range of about 30,000 to about 80,000 dalton. A high MW polymer may have a MW of about 100,000 dalton or more. In some embodiments, the oral dosage form includes a mixture of low MW polymers. In some embodiments, the oral dosage form includes a mixture of mid-weight MW polymers. In some embodiments, the oral dosage form includes a mixture of low MW and mid-weight MW polymers. In some embodiments, the oral dosage form includes a mixture of a poloxamer triblock copolymer, a polyvinylpyrrolidone (PVP) or copolymer thereof, a polyethylene glycol (PEG). In some embodiments, the preferred polymers include combinations of a poloxamer (low MW), a PVP (low or mid-weight MW), PVP copolymer (mid-weight MW) and a polyethylene glycol (low MW). Some examples of PVP polymers include K-30 (40,000-80,000 dalton), K-12 (4,000-6,000 dalton), K-15 (6,000-15,000 dalton) and the like.

Poloxamer triblock copolymers are well known by persons skilled in the art and refer to triblock copolymers having a central hydrophobic chain of polyoxypropylene (POP or polypropylene glycol, PPG) flanked by hydrophilic chains of polyoxyethylene (POE or polyethylene glycol, PEG), e.g. PEG-PPG-PEG. Examples include, Poloxamer 188, Poloxamer 237, Poloxamer 338, Poloxamer 407 and the like.

Copovidone is a copolymer of polyvinylpyrrolidone (PVP) and vinyl acetate.

In some embodiments, the at least one hydrophilic polymer comprises one or a plurality of hydrophilic polymers suitable for hot melt extrusion. In some embodiments, the hydrophilic polymer comprises a poloxamer. In some embodiments, the hydrophilic polymer comprises a polyvinylpyrrolidone or copolymer thereof. In some embodiments, the hydrophilic polymer comprises a PEG polymer. In some embodiments, the hydrophilic polymer comprises a poloxamer and a polyvinylpyrrolidone or copolymer thereof. In some embodiments, the hydrophilic polymer comprises a poloxamer and a PEG polymer. In some embodiments, the hydrophilic polymer comprises a PEG polymer and a polyvinylpyrrolidone polymer or copolymer thereof. In some embodiments, the hydrophilic polymer comprises a poloxamer, polyvinylpyrrolidone, a polyvinylpyrrolidone copolymer, a PEG polymer and any combination thereof. In some embodiments, the poloxamer is Poloxamer 188. In some embodiments, the polyvinylpyrrolidone or copolymer thereof is povidone or copovidone. In some embodiments, the polyvinylpyrrolidone or copolymer thereof includes povidone and copovidone. In some embodiments, the PEG polymer is PEG 4000.

The one or more hydrophilic polymers comprise about 15 wt % to about 60 wt % total weight of the final oral dosage form. In some embodiments, the oral dosage form comprises one or more hydrophilic polymers at about 15 wt % to about 50 wt %, about 15 wt % to about 40 wt %, about 15 wt % to about 30 wt %, about 15 wt % to about 25 wt %, about 15 wt % to about 20 wt %, about 20 wt % to about 35 wt %, about 25 wt % to about 50 wt %, or about 25 wt % to about 30 wt % of the total weight of the final oral dosage form. In some embodiments, the oral dosage form comprises one or more hydrophilic polymers at about 15 wt %, 16 wt %, 17 wt %, 18 wt %, 19 wt %, 20 wt %, 21 wt %, 22 wt %, 23 wt %, 24 wt %, 25 wt %, 26 wt %, 27 wt %, 28 wt %, 29 wt %, 30 wt %, 31 wt %, 32 wt %, 33 wt %, 34 wt %, 35 wt %, 36 wt %, 37 wt %, 38 wt %, 39 wt %, 40 wt %, 41 wt %, 42 wt %, 43 wt %, 44 wt %, 45 wt %, 46 wt %, 47 wt %, 48 wt %, 49 wt %, 50 wt %, 51 wt %, 52 wt %, 53 wt %, 54 wt %, 55 wt %, 56 wt %, 57 wt %, 58 wt %, 59 wt % or about 60 wt % of the total weight of the final oral dosage form.

The API, i.e. eltrombopag choline, is present in the dosage form at a concentration of about 5 wt % to about 30 wt % of the total weight of the dosage form. In some embodiments, the oral dosage form comprises API at about 10 wt % to about 25 wt %, about 10 wt % to about 20 wt %, from about 10 wt % to about 15 wt % of the total weight of the final oral dosage form. In some embodiments, the API comprises about 5 wt %, 5.5 wt %, 6 wt %, 6.5 wt %, 7 wt %, 7.5 wt %, 8 wt %, 8.5 wt %, 9 wt %, 9.5 wt %, 10 wt %, 10.5 wt % 11 wt %, 11.5 wt % 12 wt %, 12.5 wt % 13 wt %, 13.5 wt %, 14 wt %, 14.5 wt %, 15 wt %, 15.5 wt %, 16 wt %, 16.5 wt %, 17 wt %, 17.5 wt %, 18 wt %, 18.5 wt %, 19 wt %, 19.5 wt %, 20 wt %, 20.5 wt % 21 wt %, 21.5 wt % 22 wt %, 22.5 wt % 23 wt %, 23.5 wt %, 24 wt %, 24.5 wt %, 25 wt %, 25.5 wt %, 26 wt %, 26.5 wt %, 27 wt %, 27.5 wt %, 28 wt %, 28.5 wt %, 29 wt %, 29.5 wt % or 30 wt % of the total weight of the final oral dosage form. In some embodiments, the amount of API present in the oral dosage form is equivalent to about 10 mg to about 100 mg, or about 12.5 mg, 25 mg, 50 mg, 75 mg, or 100 mg eltrombopag free acid. In some embodiments, the ratio of eltrombopag choline to hydrophilic polymer in the dosage form is about 1:1 to about 1:5, or about 1:1 to about 1:4, or about 1:2 to about 1:3 or about 1:2.

In some embodiments, the oral dosage form comprises one or more filler. Suitable fillers are selected from mannitol, i.e. powder or spray dried, microcrystalline cellulose (MCC), silicified microcrystalline cellulose (e.g. Prosolv® SMCC, combination of MCC and colloidal silicon dioxide (CSD)), anhydrous lactose, dicalcium phosphate, tricalcium phosphate, starch, pregelatinized starch, compressible sugars, calcium carbonate, dextrates (anhydrous or hydrated) and any combination thereof. Preferred fillers comprise microcrystalline cellulose, anhydrous lactose, or combinations thereof. The oral dosage form comprises one or more fillers at a concentration of about 30 wt % to about 70 wt % of the total weight of the final oral dosage form. In some embodiments, the oral dosage form comprises one or more fillers at a concentration of about 30 wt % to about 60 wt %, about 30 wt % to about 50 wt %, about 30 wt % to about 45 wt %, from about 35 wt % to about 45 wt %, about 40 wt % to about 50 wt % or about 40 wt % to about 45 wt % of the total weight of the final oral dosage form. In some embodiments, the one or more fillers comprise about 30 wt %, 31 wt %, 32 wt %, 33 wt %, 34 wt %, 35 wt %, 36 wt %, 37 wt %, 38 wt %, 39 wt %, 40 wt %, 41 wt %, 42 wt %, 43 wt %, 44 wt %, 45 wt %, 46 wt %, 47 wt %, 48 wt %, 49 wt %, 50 wt %, 51 wt %, 52 wt %, 53 wt %, 54 wt %, 55 wt %, 56 wt %, 57 wt %, 58 wt %, 59 wt %, 60 wt %, 61 wt %, 62 wt %, 63 wt %, 64 wt %, 65 wt %, 66 wt %, 67 wt %, 68 wt %, 69 wt % or about 70 wt % of the total weight of the final oral dosage form.

In some embodiments, the oral dosage form comprises one or more disintegrant. Suitable disintegrants include hydroxypropyl cellulose (HPC), carboxymethyl cellulose (CMC) including salts thereof, croscarmellose sodium, magnesium aluminum silicate, sodium starch glycolate, starch which may be a food starch including corn starch, potato starch, tapioca starch and the like, and any combination thereof. A preferred disintegrant is croscarmellose sodium. In some embodiments, the oral dosage form comprises one or more disintegrants at a concentration of about 3 wt % to about 25 wt %, about 5 wt % to about 20 wt %, or about 10 wt % to about 15 wt % of the total weight of the final oral dosage form. In some embodiments, the oral dosage form comprises one or more disintegrants at a concentration of about 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, or about 25 wt % of the total weight of the final oral dosage form.

In some embodiments, the oral dosage form comprises one or more lubricant. Suitable lubricants include magnesium stearate, stearic acid, sodium stearyl fumarate, calcium stearate, hydrogenated vegetable oil, mineral oil, talc, and glyceryl behenate, and any combination thereof. A preferred lubricant is magnesium stearate. In some embodiments, the oral dosage form comprises one or more lubricants at a concentration of about 0.05 wt % to about 2.5 wt %, about 0.1 wt % to about 2 wt %, or about 0.1 wt % to about 1 wt % of the total weight of the final oral dosage form. In some embodiments, the oral dosage form comprises one or more lubricants at a concentration of about 0.05 wt %, 0.10 wt %, 0.15 wt %, 0.2 wt %, 0.25 wt %, 0.3 wt %, 0.35 wt %, 0.4 wt %, 0.45 wt %, 0.5 wt %, 0.55 wt %, 0.6 wt %, 0.65 wt %, 0.7 wt %, 0.75 wt %, 0.8 wt %, 0.85 wt %, 0.9 wt %, 0.95 wt %, 1.0 wt %, 1.15, 1.2 wt %, 1.3 wt %, 1.4 wt %, 1.5 wt %, 1.6 wt %, 1.7 wt %, 1.8 wt %, 1.9 wt %, 2.0 wt %, 2.1 wt %, 2.2 wt %, 2.3 wt %, 2.4 wt % or about 2.5 wt % of the total weight of the final oral dosage form.

In some embodiments, the oral dosage form further includes a chelating agent, for example ethylenediaminetetraacetate (EDTA), ammonium citrate dibasic, calcium citrate, calcium disodium ethylenediaminetetraacetate (Calcium Disodium EDTA), disodium ethylenediaminetetraacetate (disodium EDTA), glycine, sodium citrate, sodium hexametaphosphate, stearyl citrate, trisodium phosphate or any combination thereof. A preferred chelator is EDTA. In some embodiments, the oral dosage form comprises one or more chelating agents at a concentration of about 0.05 wt % to about 10.0 wt %, about 0.1 wt % to about 10.0 wt %, about 1.0 wt % to about 10.0 wt %, or about 3.0 wt % to about 8.0 wt % of the total weight of the final oral dosage form. In some embodiments, the oral dosage form comprises one or more chelating agents at a concentration of about 0.05 wt %, 0.10 wt %, 0.15 wt %, 0.2 wt %, 0.25 wt %, 0.3 wt %, 0.35 wt %, 0.4 wt %, 0.45 wt %, 0.5 wt %, 0.55 wt %, 0.6 wt %, 0.65 wt %, 0.7 wt %, 0.75 wt %, 0.8 wt %, 0.85 wt %, 0.9 wt %, 0.95 wt %, 1.0 wt %, 1.1 wt %, 1.2 wt %, 1.3 wt %, 1.4 wt %, 1.5 wt %, 1.6 wt %, 1.7 wt %, 1.8 wt %, 1.9 wt %, 2.0 wt %, 2.1 wt %, 2.2 wt %, 2.3 wt %, 2.4 wt %, 2.5 wt %, 2.6 wt %, 2.7 wt %, 2.8 wt %, 2.9 wt %, 3.0 wt %, 3.1 wt %, 3.2 wt %, 3.3 wt %, 3.4 wt %, 3.5 wt %, 3.6 wt %, 3.7 wt %, 3.8 wt %, 3.9 wt %, 4.0 wt %, 4.1 wt %, 4.2 wt %, 4.3 wt %, 4.4 wt %, 4.5 wt %, 4.6 wt %, 4.7 wt %, 4.8 wt %, 4.9 wt %, 5.0 wt %, 5.1 wt %, 5.2 wt %, 5.3 wt %, 5.4 wt %, 5.5 wt %, 5.6 wt %, 5.7 wt %, 5.8 wt %, 5.9 wt %, 6.0 wt %, 6.1 wt %, 6.2 wt %, 6.3 wt %, 6.4 wt %, 6.5 wt %, 6.6 wt %, 6.7 wt %, 6.8 wt %, 6.9 wt %, 7.0 wt %, 7.1 wt %, 7.2 wt %, 7.3 wt %, 7.4 wt %, 7.5 wt %, 7.6 wt %, 7.7 wt %, 7.8 wt %, 7.9 wt %, 8.0 wt %, 8.1 wt %, 8.2 wt %, 8.3 wt %, 8.4 wt %, 8.5 wt %, 8.6 wt %, 8.7 wt %, 8.8 wt %, 8.9 wt %, 9.0 wt %, 9.1 wt %, 9.2 wt %, 9.3 wt %, 9.4 wt %, 9.5 wt %, 9.6 wt %, 9.7 wt %, 9.8 wt %, 9.9 wt %, or about 10.0 wt % of the total weight of the final oral dosage form.

In some embodiments, the oral dosage form comprises one or more antioxidant, for example butylated hydroxyanisole (BHA), butylated hydroxytoluene (BHT), sodium ascorbate, α-tocopherol, and any combination thereof.

In some embodiments, the oral dosage form may further include a solvent such as water.

In certain embodiments, the dosage form is a tablet. In some embodiments, the tablet is coated. In some embodiments, the tablet is uncoated. In certain embodiments, the dosage form comprises granules, which are uncoated or are coated. The coating is optionally pigmented. In some embodiments, the dosage forms, e.g., tablets, having different doses, i.e. 9 mg, 18 mg etc. are coated with coatings having different colors, for example for accurate identification by a pharmacist, healthcare provider or patient. Suitable coatings are selected from those comprising for example polyvinyl alcohol (PVA) or hydroxypropyl methylcellulose (hypromellose, HPMC). Examples include Opadry® 200, Opadry® II, Opadry® fx, Opadry® amb, Opaglos® 2, Opadry® tm, Opadry®, Opadry® NS, Opalux®, Opatint® and Opaspray®.

Preferred unit dosage formulations are those containing an effective dose, or an appropriate fraction thereof, of the active ingredient. Preferred dosage forms are tablets, capsules, oral suspension and granules. In some embodiments, the dosage form is a tablet. In some embodiments, the dosage form is a capsule. In some embodiments, the dosage form is in the form of granules, for example as an oral suspension with a pharmaceutically acceptable liquid or in a pharmaceutical sachet package that may be administered per se, in liquid or in food. In some embodiments, the granules maybe taste masked.

The term “wt %” refers to a weight percent of the total weight of the oral dosage form.

In some embodiments, provided is an eltrombopag choline oral dosage form having a total weight, including coating, of from about 80 gm to about 800 gm, depending on the dosage strength. In some embodiments, the dosage strength of an eltrombopag choline tablet includes an amount of eltrombopag choline equivalent to an amount of eltrombopag free acid. The eltrombopag choline oral dosage forms disclosed herein exhibit improved bioavailability, about 25% to 30%, when compared to the commercially available eltrombopag olamine oral dosage forms (Promacta®). In particular, the oral dosage forms disclosed herein exhibit at least one of an increase in Cmax and/or AUC₀₋₇₂ and/or AUC0-ice by at least 25%, or by at least 30% when orally administered to a subject under fasting conditions compared to an equivalent dose of Promacta®. In some embodiments, the oral dosage forms disclosed herein exhibit an increase in Cmax by at least 25%, or by at least 30% when orally administered to a subject under fasting conditions compared to an equivalent dose of Promacta®. In some embodiments, the oral dosage forms disclosed herein exhibit an increase in AUC0-72 by at least 25%, or by at least 30% when orally administered to a subject under fasting conditions compared to an equivalent dose of Promacta®. In some embodiments, the oral dosage forms disclosed herein exhibit an increase in AUC0-inf by at least 25%, or by at least 30% when orally administered to a subject under fasting conditions compared to an equivalent dose of Promacta®. Accordingly, the dose adjustment for a 12.5 mg eltrombopag olamine tablet or kit for suspension is the 9 mg eltrombopag choline dosage form, for example tablet of kit for suspension; the dose adjustment for a 25 mg eltrombopag olamine tablet or kit for suspension is the 18 mg eltrombopag choline dosage form, for example tablet of kit for suspension; the dose adjustment for a 50 mg eltrombopag olamine tablet is the 36 mg eltrombopag choline dosage form; the dose adjustment for a 75 mg eltrombopag olamine tablet is the 54 mg eltrombopag choline dosage form; and the dose adjustment for a 100 mg eltrombopag olamine tablet is the 72 mg eltrombopag choline dosage form.

In certain embodiments, disclosed herein is an eltrombopag choline oral dosage form comprising
from about 5 wt % to about 30 wt % of eltrombopag choline; and
from about 15 wt % to about 60 wt % of one or more hydrophilic polymers.

The eltrombopag choline oral dosage form may further comprise
from about 30 wt % to about 70 wt % of one or more fillers;
from about 3 wt % to about 25 wt % of one or more disintegrants;
from about 0.05 wt % to about 2.5 wt % of one or more lubricants; and
optionally 0.05 wt % to 10 wt % of one or more chelating agent.

In certain embodiments, disclosed herein is an eltrombopag choline oral dosage form comprising
from about 7.5 wt % to about 20 wt % of eltrombopag choline;
from about 15 wt % to about 40 wt % of one or more hydrophilic polymers;
from about 30 wt % to about 60 wt % of one or more fillers;
from about 5 wt % to about 20 wt % of one or more disintegrants;
from about 0.1 wt % to about 2 wt % of one or more lubricants; and
optionally 1.0 wt % to 10 wt % of one or more chelating agent.

In certain embodiments, disclosed herein is an eltrombopag choline oral dosage form comprising
from about 10 wt % to about 15 wt % of eltrombopag choline;
from about 13 wt % to about 20 wt % of a low MW hydrophilic polymer;
from about 2 wt % to about 6 wt % of a mid-weight MW hydrophilic polymer;
from about 40 wt % to about 50 wt % of one or more fillers;
from about 5 wt % to about 15 wt % of one or more disintegrants;
from about 1.5 wt % to about 2 wt % of one or more lubricants; and
optionally 0.5 wt % to 10 wt % of one or more chelating agent.

In certain embodiments, the total weight of the oral dosage form is from about 80 mg to about 800 mg, depending on the dosage strength. In some embodiments, an oral dosage form comprising (i.e. eltrombopag choline) the equivalent of 12.5 mg of eltrombopag free acid weighs about 50 mg to about 100 mg. In some embodiments, an oral dosage form comprising the equivalent of 25 mg of eltrombopag free acid weighs about 150 mg to about 200 mg. In some embodiments, an oral dosage form comprising the equivalent of 50 mg of eltrombopag free acid weighs about 300 mg to about 400 mg. In some embodiments, an oral dosage form comprising the equivalent of 75 mg of eltrombopag free acid weighs about 450 mg to about 600 mg. In some embodiments, an oral dosage form comprising the equivalent of 100 mg of eltrombopag free acid weighs about 600 mg to about 800 mg.

The present disclosure further encompasses processes to prepare eltrombopag choline oral dosage forms disclosed herein. In some embodiments, the processes include formation of a melted dispersion of eltrombopag choline and one or more hydrophilic polymers. In some embodiments, the processes include hot melt extrusion of the eltrombopag and one or more hydrophilic polymers.

In some embodiments, provided herein is a process for preparing an oral dosage form, comprising:
a. providing eltrombopag choline and one or more hydrophilic polymer;
b. mixing the eltrombopag choline and the one or more hydrophilic polymer to form a mixture;
c. heating the mixture to a temperature sufficient to form a molten dispersion of the eltrombopag choline and the hydrophilic polymer;
d. milling the dispersion from step (c) to provide granules (i.e. solid dispersion) comprising the eltrombopag choline and the hydrophilic polymers.

The steps in the process may be carried out by a single entity or multiple entities. In some embodiments, the one or more hydrophilic polymer is selected from a low MW polymer, for example Poloxamer 188, Povidone, PEG 4000 and any combination thereof. In some embodiments the one or more hydrophilic polymers includes a combination of Poloxamer 188, Povidone and PEG 4000. In some embodiments, the dosage form further includes a mid-weight MW hydrophilic polymer. In certain embodiments, the dosage form includes a combination of low MW and mid-weight MW polymers.

In some embodiments the granules from step (e) are admixed with a common blend to form an eltrombopag choline blend. The eltrombopag common blend may be, for example, used in the preparation of a kit for suspension or may be compressed into tablets.

In some embodiments, the common blend comprises one or more of a filler, a disintegrant, and or a lubricant. In some embodiments the blend further comprises a chelating agent. In some embodiments, the common blend includes silicified microcrystalline cellulose. In some embodiments, the common blend includes anhydrous lactose. In some embodiments, the common blend includes croscarmellose sodium. In some embodiments, the common blend includes magnesium stearate.

In some embodiments, the process further includes the steps of
e. compressing the eltrombopag choline blend of step (e) into tablets, filling a capsule shell, preparing an oral suspension or filling a sachet package with the eltrombopag choline blend of step (e); and
f. optionally, coating the tablets or capsule shell.

In some embodiments, the eltrombopag choline blend is compressed into a tablet. In some embodiments, the oral dosage form is a tablet. In some embodiments, the tablet is a coated tablet. In some embodiments, each dose comprises a coating with different pigment to differentiate between the doses.

In another embodiment, the present disclosure encompasses eltrombopag choline oral dosage forms for the treatment of idiopathic thrombocytopenic purpura, thrombocytopenia and aplastic anemia. In some embodiments, the treatment is independent of food intake.

The present disclosure also provides methods of treating idiopathic thrombocytopenic purpura, thrombocytopenia and aplastic anemia; comprising administering a therapeutically effective amount of an eltrombopag choline oral dosage form as disclosed in the present disclosure, to a subject suffering from idiopathic thrombocytopenic purpura, thrombocytopenia and aplastic anemia, or otherwise in need of the treatment. In some embodiments, the method of treatment is independent of food and the oral dosage forms may be administered to a subject in a fed state or in a fasted state.

In some embodiments, the methods of treating comprise administering a therapeutically effective amount of an eltrombopag choline oral dosage form as disclosed in the present disclosure in combination with a second therapeutic agent, to a subject suffering from idiopathic thrombocytopenic purpura, thrombocytopenia and aplastic anemia, or otherwise in need of the treatment.

For the foregoing embodiments, each embodiment disclosed herein is contemplated as being applicable to each of the other disclosed embodiments. For example, the features recited in the method embodiments can be used in the granules (i.e. solid dispersion), pharmaceutical composition, package, and use embodiments described herein and vice versa.

Having described the disclosure with reference to certain preferred embodiments, other embodiments will become apparent to one skilled in the art from consideration of the specification. The disclosure is further illustrated by reference to the following examples describing in detail the preparation of the composition and methods of use of the disclosure. It will be apparent to those skilled in the art that many modifications, both to materials and methods, may be practiced without departing from the scope of the disclosure.

### EXAMPLES

### Example 1: Manufacture of Eltrombopag Choline Tablets, 25 mg and 50 mg

Eltrombopag choline was prepared as described in International Patent Publication No. WO20190071111, the entirety of which is incorporated herein by reference.

The drug product manufacturing process includes the following steps:

### 1. Granulation

### 2. Common Blend

### 3. Compression (Core Tablets)

### 4. Color Coating

### 5. Packaging in Bottles

Granulation: In the following order, the raw materials were added into an 8 quart V-blender and blended at 25 RPMs for 5 minutes: Eltrombopag choline and hydrophilic polymers. The hydrophilic polymers used were Poloxamer 188, NF (Kolliphor® P 188), Copovidone, NF (Kollidon® VA 64 Fine), Polyethylene Glycol 4000, NF (Powder) and Povidone, NF (Kollidon® 12 PF).

After blending, the mixed material was passed through a #30 mesh screen and blended again for 5 minutes at 25 RPMs in an 8 quart V-Blender. The screened blended material was then fed into a Leistritz Nano-16 Extruder to yield a hot melt extrusion utilizing the process settings set forth in Table 1.

TABLE 1
Hot melt extrusion process settings

| | |
|---|---|
| Zone Temperatures | Zone 1: 80° C. |
| | Zone 2: 100° C. |
| | Zone 3: 110° C. |
| | Zone 4: 125° C. |
| Feeding Rate | manually fed |
| Extruder Speed | 150 rpm |
| Conveyor Belt Speed | 28.0 rpm |
| Melt Pressure | 67 psi |
| Melt Temperature | 135° C. |
| Chiller Temperature | 10° C. |

The acceptable extrudates were collected and milled. A Fitzmill with a #27 screen size and a knife forward set up with an impeller speed of 2400 rpm was used to granulize the extrudates. The milled granules were collected and placed into an 8 quart V blender and blended at 25 RPMs for 5 minutes. This yielded the final state of the eltrombopag choline granules, or “eltrombopag solid dispersion”.

Common Blend: Silicified microcrystalline cellulose, anhydrous lactose, and croscarmellose sodium were screened through a #30 mesh screen and collected. Magnesium stearate was passed through a #40 mesh screen and collected.

Eltrombopag choline granules along with the screened silicified microcrystalline cellulose, anhydrous lactose, and croscarmellose sodium were loaded into an 8 quart V-blender and blended at 25 RPMs for 10 minutes. The screened magnesium stearate was added to the V-blender and the ingredients blended for 5 minutes at 25 RPMs. This yielded the final state of the eltrombopag choline blend.

Compression (Core Tablets): Utilizing a tablet press, the eltrombopag choline blend was compressed to yield the core tablets of the 25 mg and 50 mg strengths of the drug product.

The 25 mg and 50 mg tablet press set-up parameters are shown in Table 2.

TABLE 2
Tablet press set-up parameters

| | 25 mg | 50 mg |
|---|---|---|
| Die number | 95844 | 94955 |
| Punch size | 0.2775” × 0.5220” | 0.3150” × 0.6299” |
| Punch shape | Oval shape | Oval shape |
| | Upper: embossed “WPI”. | Plain upper and lower |
| | Lower: embossed “100” | |
| Tablet weight | 218 mg (202-234 mg) | 435 mg (402-468 mg) |
| Weight of 10 tablets | 2.18 g (2.07-2.29 g) | 4.35 g (4.13-4.57 g) |
| Thickness | 0.16” (0.13-0.19”) | 0.2” (0.17-0.23”) |
| Hardness | 5 kp (3-7 kp) | 5 kp (3-7 kp) |
| Friability | NMT 1% | NMT 1% |
| Tablet press speed | 18-72 rpm (+10%) | 18-72 rpm (+10%) |

The compression of the 25 mg strength core tablets yielded brown oval tablets with dark brown spots that were embossed with “WPI” on one side and with “100” on the other. For the 50 mg strength, the compression yielded core tablets that were brown oval tablets with dark brown spots and no embossing; i.e., tablets are plain on both sides.

Color Coating: Water was dispensed into a clean, stainless steel tank with a mixer. The mixer was turned on to create a vortex and the film coating material, Opadry II Complete Film Coating System 85F250106 Red was slowly added into the clean, stainless steel tank and mixed for 30 minutes or until homogenously dispersed to yield the color coating suspension. It was left mixing throughout the coating process. Using a Vector Pan Coater with a 1.3 Liter pan insert with a gun-to-bed distance of 2 to 5 inches, the 25 mg and 50 mg core tablets were coated until the target weight of 100 tablets was achieved (2.5 wt % to 3.5 wt %), following the conditions shown in Table 3:

TABLE 3
Tablet Coating Conditions

| Parameter | Target | Range | QEP | Tolerance |
|---|---|---|---|---|
| Exhaust Temp (° C.) | 43 | 40-50 | 30-90 | ±5° C. |
| Atomization (psi) | 20 | 15-30 | 15-30 | ±5% full scale |
| Process Air Volume (cfm) | 25 | 15-30 | 15-30 | ±10% |
| Pan Speed (rpm) | 15 | 10-30 | 10-30 | ±5% full scale |
| Spray Rate g/min | record | 3-12 | N/A | N/A |
| Inlet Air Temp (° C.) | record | record | 30-90 | ±5° C. |

After color coating, the core tablets were allowed to dry for 10 minutes under the conditions shown in Table 4.

TABLE 4
Drying conditions

| Parameter | Range |
|---|---|
| Pan speed | 10-15 RPM |
| Inlet Air Temp | record |
| Process Air Volume | 15-30 cfm |
| Exhaust Temp | 30-50° C. |

This yielded the bulk drug product for the 25 mg and 50 mg strengths, which were collected and packaged.

The 25 mg and 50 mg tablets contain eltrombopag choline equivalent to 25 mg and 50 mg of eltrombopag free acid, respectively.

Packaging in Bottles: The 25 mg and 50 mg eltrombopag choline tablets were packaged in 30 count bottles.

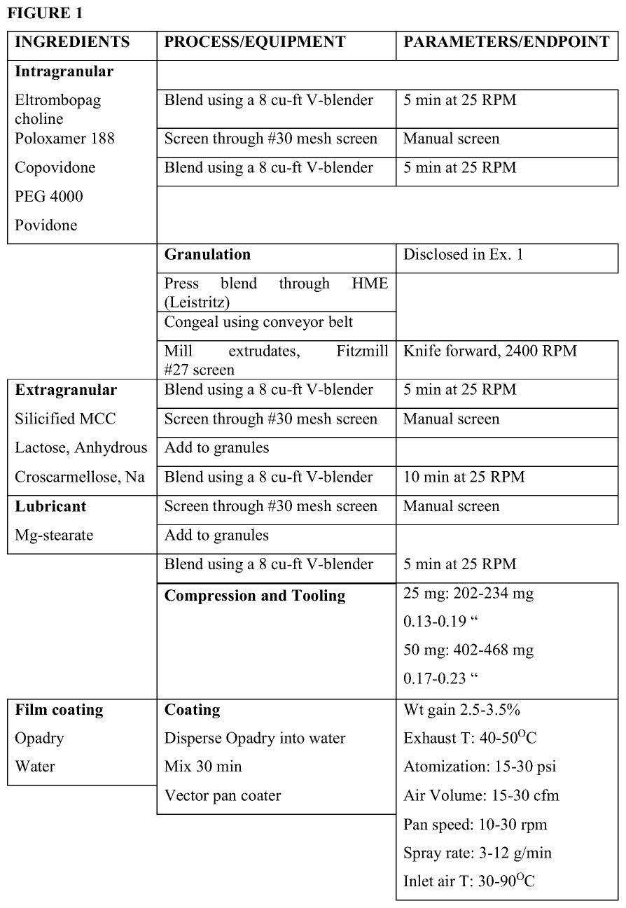

Manufacturing Process Flow: The flow diagram for the manufacturing process of representative batches of eltrombopag choline is shown in FIG. 1.

Exemplary formulations are provided in Tables 5A-5D. Values are provided in milligrams (mg), unless otherwise noted.

TABLE 5A
Eltrombopag choline formulations

| BATCH | 91 | 92 | 94 | 16 | 18 |
|---|---|---|---|---|---|
| Eltrombopag Choline | 61.66 | 61.66 | 61.66 | 61.66 | 61.66 |
| Poloxamer 188 NF (Kolliphor ® P 188) | 47.09 | 62.78 | 30 | 15 | 15 |
| Polyethylene Glycol 4000, NF(Powder) | 47.08 | 62.78 | 31.51 | 15 | 15 |
| Povidone, NF (Kollidon ® 12 PF) | | | 30.83 | 45 | 30 |
| Copovidone, NF (Kollidon ® VA 64 Fine) | 94.17 | 62.78 | 31 | 45 | 30 |
| Silicified MCC (Prosolv ® SMCC 90) | 147.5 | 147.5 | 147.5 | 148.15 | 148.15 |
| Lactose Anhydrous | 50 | 50 | 50 | 50 | 50 |
| Croscarmellose Sodium | 50 | 50 | 50 | 50 | 50 |
| Colloidal Silicon Dioxide, NF | | | | 4.35 | 4.35 |
| (Aerosil ® 200 Pharma) | | | | | |
| Magnesium Stearate | 2.5 | 2.5 | 2.5 | 2.5 | 2.5 |
| Total Tablet Weight | 500 | 500 | 435 | 437 | 407 |

TABLE 5B
Eltrombopag choline formulations

| BATCH | 85 | 86 | 88 | 89 | 90 |
|---|---|---|---|---|---|
| Eltrombopag Choline | 61.66 | 61.66 | 61.66 | 61.66 | 61.66 |
| Poloxamer 188 NF (Kolliphor P 188) | 94.17 | | 47.09 | 62.78 | 47.09 |
| Polyethylene Glycol 4000, NF (Powder) | | 94.17 | 47.08 | 62.78 | 47.08 |
| Povidone, NF (Kollidon ® 12 PF) | | 94.17 | | | 47.08 |
| Copovidone, NF (Kollidon ® VA 64 Fine) | 94.17 | | 94.17 | 62.78 | 47.09 |
| Silicified MCC (Prosolv ® SMCC 90) | 147.5 | 147.5 | 147.5 | 147.5 | 147.5 |
| Lactose Anhydrous | 50 | 50 | 50 | 50 | 50 |
| Croscarmellose Sodium | 50 | 50 | 50 | 50 | 50 |
| Colloidal Silicon Dioxide, NF | | | | | |
| (Aerosil ® 200 Pharma) | | | | | |
| Magnesium Stearate | 2.5 | 2.5 | 2.5 | 2.5 | 2.5 |
| Total Tablet Weight | 500 | 500 | 500 | 500 | 500 |

TABLE 5C
Eltrombopag choline formulations

| BATCH | 78 | 79 | 80 |
|---|---|---|---|
| Eltrombopag Choline | 61.66 | 61.66 | 61.66 |
| Poloxamer 188 NF (Kolliphor P 188) | 238.34 | 238.34 | |
| Polyethylene Glycol 4000, NF (Powder) | | | 238.34 |
| Povidone, NF (Kollidon ® 12 PF) | | | 200.00 |
| Copovidone, NF (Kollidon ® VA 64 Fine) | | 200 | |
| Soluplus ®* | 200 | | |
| Silicified MCC (Prosolv ® SMCC 90) | 147.5 | 147.5 | 147.5 |
| Lactose Anhydrous | 50 | 50 | 50 |
| Croscarmellose Sodium | 50 | 50 | 50 |
| Magnesium Stearate | 2.5 | 2.5 | 2.5 |
| Total Tablet Weight | 500 | 500 | 500 |

*Polyvinyl caprolactam-polyvinyl acetate-polyethylene glycol graft copolymer

TABLE 5D
Eltrombopag choline formulations

| | | Dosage Strength | |
|---|---|---|---|
| | %/Core | 25 mg | 50 mg |
| Eltrombopag Choline | 13.12 | 30.8 | 61.7 |
| Poloxamer 188 NF (Kolliphor ® P 188) | 3.19 | 7.5 | 15.0 |
| Copovidone, NF (Kollidon ® VA 64 Fine) | 3.19 | 7.5 | 15.0 |
| Polyethylene Glycol 4000, NF (Powder) | 6.38 | 15.0 | 30.0 |
| Povidone, NF (Kollidon ® 12 PF) | 6.38 | 15.0 | 30.0 |
| Edetate Disodium Dihydrate, USP | 7.02 | 16.5 | 33.0 |
| Silicified Microcrystalline Cellulose | 37.43 | 88.0 | 175.9 |
| (Prosolv ® SMCC 90), NF | | | |
| Anhydrous Lactose NF | 10.64 | 25.0 | 50.0 |
| Croscarmellose Sodium, NF | 10.64 | 25.0 | 50.0 |
| Magnesium Stearate, NF | 1.00 | 2.4 | 4.7 |
| Silicon Dioxide, NF (Syloid ® 244FP) | 1.00 | 2.4 | 4.7 |
| Core Tablet | 100.0% | 235.0 | 470.0 |
| Film Coating | | | |
| Opadry II 85F205003 Blue | — | — | — |
| Opadry White 00F580001 | — | — | — |
| Opadry II 85F250106 Red | — | 7.0 | 14.1 |
| Opadry 02B130008 Orange | — | — | — |
| Purified Water, USP | — | — | — |
| | — | 242.0 | 484.1 |

Tablets, which have been dose adjusted for the improved bioavailability (BA) of eltrombopag choline dosage forms, were manufactured using the components and quantities shown in Table 5D. The dose adjustment for 12.5 mg eltrombopag olamine is the 9 mg eltrombopag choline dosage form; the dose adjustment for 25 mg eltrombopag olamine is the 18 mg eltrombopag choline dosage form; the dose adjustment for 50 mg eltrombopag olamine is the 36 mg eltrombopag choline dosage form; the dose adjustment for 75 mg eltrombopag olamine is the 54 mg eltrombopag choline dosage form; and the dose adjustment for 100 mg eltrombopag olamine is the 72 mg eltrombopag choline dosage form.

### Example 2: In Vitro Dissolution Testing of Eltrombopag Choline Tablets

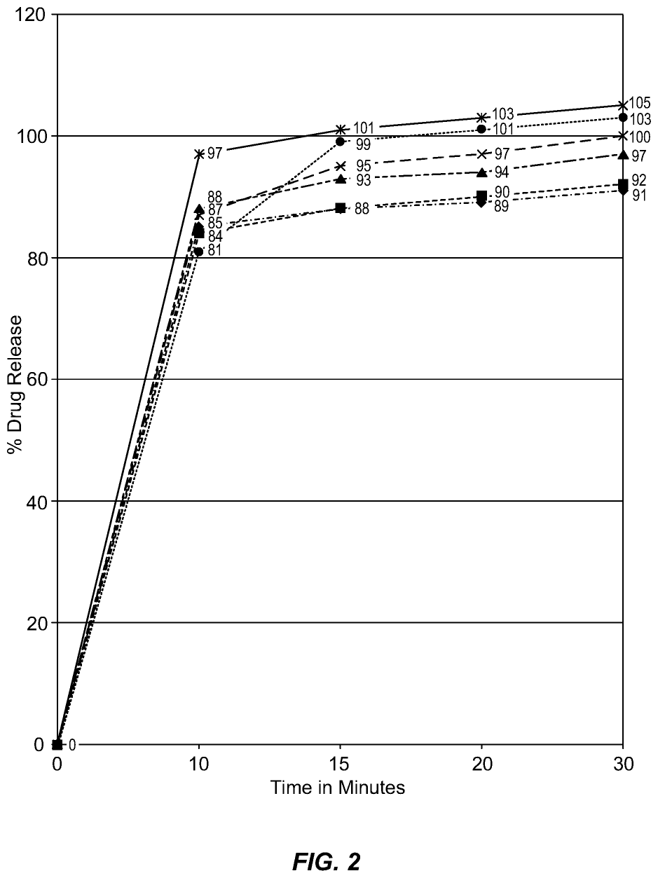

In vitro dissolution studies were performed for the 9 mg, 18 mg, 25 mg, 36 mg, 50 mg, and 54 mg eltrombopag choline tablets. Dissolution was performed in a USP apparatus, 900 mL, pH 6.8 no surfactant, 50 RPM paddle in peak vessel. Results are provided in Table 6 and FIG. 2. In FIG. 2, diamonds represent the 25 mg dose tablets, squares represent the 50 mg dose tablets, triangles represent the 9 mg dose tablets, x's represent the 18 mg dose tablets, stars represent the 36 mg dose tablets and the circles represent the 54 mg dose tablets.

TABLE 6
in vitro drug release (%) over time

| Lot | BA | | Dose adjusted | | | |
|---|---|---|---|---|---|---|
| Time/dose | 25 mg | 50 mg | 9 mg | 18 mg | 36 mg | 54 mg |
| 0 | 0 | 0 | 0 | 0 | 0 | 0 |
| 10 min | 85 | 84 | 88 | 87 | 97 | 81 |
| 15 min | 88 | 88 | 93 | 95 | 101 | 99 |
| 20 min | 89 | 90 | 94 | 97 | 103 | 101 |
| 30 min | 91 | 92 | 97 | 100 | 105 | 103 |

### Example 3: Bioavailability of Eltrombopag Choline Tablets, 25 mg and 50 mg

Study Title: An open label, randomized, single dose, four-way crossover comparative bioavailability study of eltrombopag choline tablets, 25 mg and 50 mg in healthy human, adult males and females of non-childbearing potential under fasting and fed conditions.

The bioavailability studies were conducted in 24 healthy subjects (adult males and non-pregnant females) for the evaluation of pharmacokinetic profiles and food effect.

The 25 mg and 50 mg eltrombopag choline tablets, described in Example 1, supra, were compared to the commercially available 50 mg eltrombopag olamine 50 mg (Promacta®). Promacta® tablets comprise a core with eltrombopag olamine and the inactive ingredients magnesium stearate, mannitol, microcrystalline cellulose, povidone and sodium starch glycolate.

Test Product 1 (A): Eltrombopag Tablets, 25 mg (each tablet contains eltrombopag choline equivalent to 25 mg of eltrombopag free acid) under fasting conditions, Lot #02A.

Test Product 2 (B): Eltrombopag Tablets, 50 mg (each tablet contains eltrombopag choline equivalent to 50 mg of eltrombopag free acid) under fasting conditions, Lot #02A.

Test Product 2 (C): Eltrombopag Tablets, 50 mg (each tablet contains eltrombopag choline equivalent to 50 mg of eltrombopag free acid) under fed conditions, Lot #02A.

Reference Product (D): Promacta® Tablets, 50 mg (each tablet contains eltrombopag olamine, equivalent to 50 mg of eltrombopag free acid) under fasting conditions, Lot #L97G

The primary objective was to determine pharmacokinetic parameters

maximum observed concentration (Cmax)

area under the plasma concentration-time (AUC) from time 0 to the time of the last measurable plasma concentration (72 h) (AUC₀₋₇₂)

AUC extrapolated to infinity (AUC_{0-∞})

Subjects were randomly assigned to the four treatment groups in the four periods of the study as per SAS® software (Version 9.4) generated randomization schedule. Each Subject received one study product (i.e., test product 1 (A) or test product 2 (B) under fasting condition or test product 2 (C) under fed condition or reference product (D)) in one of the periods as per the randomization schedule. The order of administration was according to a four-treatment (A, B, C and D), four-sequence (Sequence 1=ADBC, Sequence 2=BACD, Sequence 3=CBDA and Sequence 4=DCAB) randomization schedule.

For Test product 1 (A), Test product 2 (B) and Reference product (D) (Under Fasting conditions):

In each study period, following an overnight fast of at least 10 hours, a single oral dose of investigational product (1×25 mg tablet of Test product 1 (A) or 1×50 mg tablet of Test product 2 (B) or 1×50 mg tablet of reference product (D)) was administered with 240 mL of water at ambient temperature as per the randomization schedule under fasting condition.

For Test Product 2 (C) (Under Fed Conditions):

In each study period, following an overnight fast of at least 10 hours, subjects were served standard test meal (standardized high calorie, high fat breakfast) 30 minutes prior to scheduled administration of the investigational medicinal product. Exactly 30 minutes after start of the test meal, a single oral dose of investigational medicinal product (1×50 mg tablet of Test product 2 (C)) was administered with 240 mL of water at ambient temperature as per the randomization schedule under fed conditions.

A qualified and trained designee administered the oral doses as per the study protocol. The subjects were instructed not to chew or crush the tablet but to consume it whole with a specified quantity of water. The subject's oral cavity was checked to confirm the complete medication and fluid consumption after dosing and also further confirmed by analysis of plasma samples for drug concentrations. Complete accountability of investigational products was maintained in the pharmaceutical services records for its receipt and usage in the study.

Safety assessments were done at screening, at regular intervals during the study and at the end of the study and were based on clinical observations, laboratory investigations, and evaluation of the AE(s) observed during the course of the study. Screening of study subjects was performed under a general screening consent for assessment of the subjects' health to evaluate their eligibility for participation. All assessments previously performed under generic screening consent were used for enrollment purposes as long as a study-specific consent was signed prior to any other study-specific study procedure or assessment.

Standard protocols were followed for blood collection and processing for the pharmacokinetic (PK) analyses.

Results:

Statistical analysis was performed on pharmacokinetic data of Eltrombopag using the SAS package (SAS® Institute Inc., USA, Version 9.4).

Descriptive Statistics: Number of observations (N), arithmetic mean, standard deviation (SD), coefficient of variation (CV %), minimum, median, maximum and geometric mean were estimated.

Analyses of Variance: The log-transformed pharmacokinetic parameters AUC0-72, AUC0-inf and Cmax of Eltrombopag were analyzed using a Linear mixed-effects model (Proc Mixed of SAS®) with the main effects of treatment, period and sequence as fixed effects and subjects nested within sequence as random effect.

A separate Mixed ANOVA model was used to analyze each of the parameters.

Main effects were tested at the 0.05 level of significance against the residual error (mean square error/MSE) from the ANOVA as the error term. Each analysis of variance included calculation of least-square means, the difference between the adjusted formulation means and the standard error associated with the difference. The above analyses were done using the appropriate SAS® procedure.

Test Product-1 (A) Under Fasting Condition vs. Reference Product (D) Under Fasting Condition. PK data is shown in Table 7. Test Product-2 (B) Under Fasting Condition vs. Reference Product (D) Under Fasting Conditions. PK data is shown in Table 8. Test Product-2 (C) Under Fed Condition vs. Test Product-2 (B) Under Fasting Conditions. PK data is shown in Table 9.

TABLE 7
PK data from comparing test product A to reference D under fasting conditions

| Parameter | TestGeoLSM A | RefGeoLSM D | Ratio % | 90% CI | ISCV |
|---|---|---|---|---|---|
| Ln Cₘₐₓ | 3876.88 | 5428.68 | 71.41 | 58.45-87.26 | 41.34 |
| Ln AUC₀₋₇₂ | 35609.50 | 49890.77 | 71.37 | 61.14-83.32 | 31.26 |
| Ln AUC_{0-inf} | 37738.78 | 53249.49 | 70.87 | 60.55-82.96 | 31.82 |

TABLE 8
PK data from comparing test product B to reference D under fasting conditions

| Parameter | TestGeoLSM B | RefGeoLSM D | Ratio % | 90% CI | ISCV |
|---|---|---|---|---|---|
| Ln Cmax | 7139.89 | 5428.68 | 131.52 | 98.96-174.79 | 61.13 |
| Ln AUC0-72 | 66952.76 | 49890.77 | 134.20 | 106.71-168.77 | 47.73 |
| Ln AUC0-inf | 72900.58 | 53249.49 | 136.90 | 109.88-170.57 | 45.57 |

TABLE 9
PK data comparing test product C to test product B under fasting conditions

| Parameter | TestGeoLSM C | TestGeoLSM B | Ratio % | 90% CI | ISCV |
|---|---|---|---|---|---|
| Ln Cₘₐₓ | 4374.64 | 7187.07 | 60.87 | 44.96-82.40 | 65.84 |
| Ln AUC₀₋₇₂ | 43869.83 | 67396.71 | 65.09 | 50.66-83.64 | 52.78 |
| Ln AUC_{0-inf} | 46996.35 | 73406.91 | 64.02 | 50.59-81.02 | 49.15 |

Analyses and Results:

Log transformation (Ln) of exposure measurements are provided in the tables, above. Geometric least square mean (GeoLSM) units are ng/mL for Cmax and (ng)(hr)/mL for AUC. The 90% CI for the ratio of geometric means between test and reference products is provided. An absence of food effect on BA is established when the 90% CI falls within the equivalence limits of 80%-125% (as required for by the FDA). Intra-subject coefficients of variation (ISCVs) were provided.

The eltrombopag choline dosage forms disclosed herein show improved bioavailability compared to Promacta®.

The Promacta® label states that a standard high-fat breakfast “significantly decreased plasma eltrombopag AUC_{0-inf} by approximately 59% and Cmax by 65%.” However, as can be seen in Table 8 below, the eltrombopag choline 50 mg tablets had less of a decline in both plasma AUC_{0-inf} (approximately 36%) and Cmax (39%) in fed subjects. Thus, the eltrombopag choline dosage forms did not show as much of a food effect as the commercialized eltrombopag olamine product.

TABLE 10
Food effect comparison for the 50 mg eltrombopag choline tablets

| | | | | Promacta ® Tablets, |
|---|---|---|---|---|
| | Tablets, 50 mg | Tablets, 50 mg | Tablets, 50 mg | 50 mg. % decrease (fed) |
| Parameter | Fasting (B) | Fed (C) | % decrease (fed) | (taken from label) |
| Ln Cₘₐₓ | 7187 | 4375 | 39.1% | 65% |
| Ln AUC₀₋ₜ | 67397 | 43870 | 34.9% | Not available |
| Ln AUC_{0-inf} | 73407 | 46996 | 35.9% | 59% |

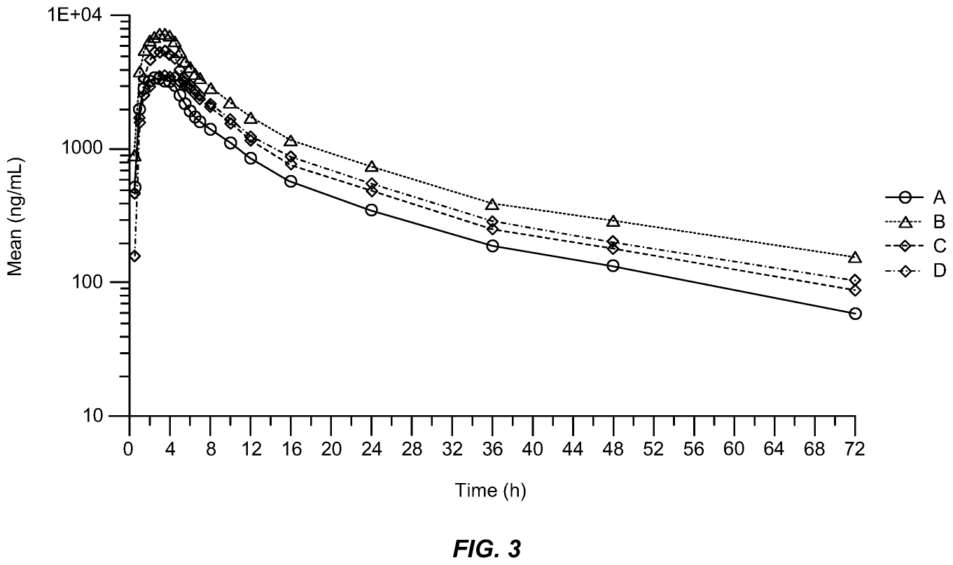

FIG. 3 shows the semi-log plot of mean plasma eltrombopag concentrations vs. time. The ovals represent test product A, the triangles represent test product B, the upper line with diamonds represents test product C and the lower line with diamonds represents reference product D.

### Example 4: Methods of Treating

The eltrombopag choline dosage forms are shown to be effective in treating disorders in which stimulation of the proliferation and differentiation of megakaryocytes is indicated, for example:

In adult and pediatric patients 1 year and older with chronic immune thrombocytopenia (ITP) who have had an insufficient response to corticosteroids, immunoglobulins, or splenectomy. It is to be used in patients with ITP whose degree of thrombocytopenia and clinical condition increase the risk for bleeding;

In patients with chronic hepatitis C to allow the initiation and maintenance of interferon based therapy. It should be used only in patients with chronic hepatitis C whose degree of thrombocytopenia prevents the initiation of interferon-based therapy or limits the ability to maintain interferon-based therapy;

In combination with standard immunosuppressive therapy for the first-line treatment of adult and pediatric patients 2 years and older with severe aplastic anemia.

For the treatment of patients with severe aplastic anemia who have had an insufficient response to immunosuppressive therapy.

All patent documents and publications are herein incorporated by reference to the same extent as if each individual publication was specifically and individually incorporated by reference. The invention illustratively described herein suitably may be practiced in the absence of any element(s) not specifically disclosed herein. Thus, for example, in each instance herein any of the terms “comprising”, and “consisting of” may be replaced with either of the other two terms. For the foregoing embodiments, each embodiment disclosed herein is contemplated as being applicable to each of the other disclosed embodiments. For instance, the elements recited in the method embodiments can be used in the pharmaceutical composition, package, and use embodiments described herein and vice versa. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be contemplated by those skilled in the art, and that such modifications and variations are considered to be within the scope of this invention as defined by the appended claims.

1. An oral dosage form comprising eltrombopag choline granulate, wherein the eltrombopag choline granulate comprises eltrombopag choline and at least one hydrophilic polymer.

2. The oral dosage form of claim 1, wherein the at least one hydrophilic polymer has a molecular weight in the range of 2,000 to about 10,000 daltons.

3. The oral dosage form of claim 2, wherein the at least one hydrophilic polymer is selected from a poloxamer, polyvinylpyrrolidone, a polyethylene glycol or any combination thereof.

4. The oral dosage form of claim 1, comprising a hydrophilic polymer having a molecular weight of about 30,000 to about 80,000 daltons.

5. The oral dosage form claim 1, wherein a total amount of the at least one hydrophilic polymer present in the dosage form is about 15 wt % to about 60 wt %, of the total weight of the dosage form.

6. The oral dosage form of claim 1, wherein the eltrombopag choline is present at a concentration of about 5 wt % to about 30 wt % of the total weight of the dosage form.

7. The oral dosage form of claim 1, wherein the ratio of eltrombopag choline to hydrophilic polymer in the dosage form is about 1:1 to about 1:5, or about 1:1 to about 1:4, or about 1:2 to about 1:3, or about 1:2.

8. The oral dosage form of claim 1, further comprising at least one excipient selected from a disintegrant, a lubricant, a filler, a chelating agent or any combination thereof.

9. The oral dosage form of claim 8, comprising a disintegrant, the disintegrant selected from hydroxypropyl cellulose (HPC), carboxymethyl cellulose (CMC) and salts thereof, croscarmellose sodium, magnesium aluminum silicate, sodium starch glycolate, a starch or any combination thereof.

10. The oral dosage form of claim 8, wherein the disintegrant is present in the dosage form at a concentration of about 3 wt % to about 25 wt %, of the total weight of the dosage form.

11. The oral dosage form of claim 8, comprising a lubricant, the lubricant selected from magnesium stearate, stearic acid, sodium stearyl fumarate, calcium stearate, hydrogenated vegetable oil, mineral oil, talc, and glyceryl behenate, or any combination thereof.

12. The oral dosage form of claim 11, wherein the lubricant is present in the dosage form at a concentration of about 0.05 wt % to about 2.5 wt %, of the total weight of the dosage form.

13. The oral dosage form of claim 8, comprising a filler, the filler selected from one or more of mannitol, microcrystalline cellulose, silicified microcrystalline cellulose, anhydrous lactose, dicalcium phosphate, tricalcium phosphate, starch, pregelatinized starch, compressible sugars, calcium carbonate or any combination thereof.

14. The oral dosage form of claim 13, wherein the filler is present in the dosage form at a concentration of about 30 wt % to about 70 wt %, of the total weight of the dosage form.

15. The oral dosage form of claim 8, further comprising a disintegrant, a lubricant, and a filler.

16. The oral dosage form of claim 15, comprising
from about 5 wt % to about 30 wt % of eltrombopag choline;
from about 15 wt % to about 60 wt % of at least one hydrophilic polymers;
from about 30 wt % to about 70% of one or more fillers;
from about 3 wt % to about 25 wt % of one or more disintegrants;
from about 0.05 wt % to about 2.5 wt % of one or more lubricants; and
optionally from about 0.05 wt % to about 10 wt % of at least one chelating agent.

17. The oral dosage form of claim 15, comprising
from about 10 wt % to about 15 wt % of eltrombopag choline;
from about 15 wt % to about 30 wt % of one or more hydrophilic polymers;
from about 40 wt % to about 50 wt % of one or more fillers;
from about 10 wt % to about 15 wt % of one or more disintegrants;
from about 0.1 wt % to about 1 wt % of one or more lubricants; and optionally
from about 0.5 wt % to about 10 wt % of at least one chelating agent.

18. The oral dosage form of claim 1, in the form of a tablet, granules or a suspension.

19. The oral dosage form of claim 18 in the form of a tablet.

20. The oral dosage form of claim 18, in the form of granules.

21. The oral dosage form of claim 18, in the form a suspension.

22. The oral dosage form of claim 18, comprising 9 mg eltrombopag choline, 18 mg eltrombopag choline, 25 mg eltrombopag choline, 36 mg eltrombopag choline, 50 mg eltrombopag choline, 54 mg eltrombopag choline or 72 mg eltrombopag choline.

23. A method of treating a subject having a disorder in which stimulating the proliferation and differentiation of megakaryocytes provides a beneficial effect on the subject, the method comprising administering an oral dosage form of claim 1 to a subject suffering from the disorder.

24. The method of claim 23, wherein the oral dosage form is administered to the subject without regard to food intake.

25. The method of claim 23, wherein the dosage form exhibits at least one of an increase in Cmax, AUC0-72 or AUC0-inf by at least 25%, or by at least 30% when orally administered to a subject under fasting conditions compared to an equivalent dose of commercially available eltrombopag olamine.

26. The method of claim 23, wherein the disorder is selected from idiopathic thrombocytopenic purpura, thrombocytopenia and aplastic anemia.

27. A process for preparing an eltrombopag choline oral dosage form, comprising:
a. providing eltrombopag choline and at least one hydrophilic polymer;
b. mixing the eltrombopag choline and the at least one hydrophilic polymer to form a mixture;
c. heating the mixture of step (b) to a temperature sufficient to form a molten dispersion of the eltrombopag choline and the at least one hydrophilic polymer;
d. milling the composition from step (c)
thereby providing granules comprising the eltrombopag choline and the at least one hydrophilic polymer.

28. The process of claim 27, further comprising the steps of
mixing the granules from step (d) with an excipient to form an eltrombopag choline blend;
compressing the eltrombopag choline blend into tablets; and
optionally, coating the tablets.

29. The process of claim 28, wherein the excipient comprises a mixture of filler, disintegrant, lubricant and optionally a chelating agent.

30. An eltrombopag choline dosage form manufactured by the process of claim 27.
